(19) United States
(12) Patent Application Publication
(10) Pub. No.: US 2009/0105237 A1
(43) Pub. Date: Apr. 23, 2009

(54) Cooling compounds

(76) Inventors: Karen Ann Bell, Loveland, OH (US); Stefan Michael Furrer, Cincinnati, OH (US); Christophe C. Galopin, Chesterfield, VA (US); Pablo Victor Krawec, Cincinnati, OH (US); Jay Patrick Slack, Loveland, OH (US)

Correspondence Address: CURATOLO SIDOTI CO., LPA, 24500 CENTER RIDGE ROAD, SUITE 280, CLEVELAND, OH, 44145, US

(21) Appl. No.: 11/920,813

(22) PCT Filed: May 24, 2006
(86) PCT No.: PCT/CH2006/000270

§ 371 Date: Feb. 19, 2008

Related U.S. Application Data
(60) Provisional application No. 60/685,548, filed on May 27, 2005.

(51) Int. Cl. A61K 31/167; A61K 31/4965; A61K 31/357; C07D 295/14; C07C 229/40; C07C 237/24; A61P 17/00; C07C 229/36; C07D 317/30; A61K 31/216; A61K 31/5375

(52) U.S. Cl. 514/231.2; 514/255.03; 514/466; 514/255.01; 514/613; 514/529; 514/538; 514/616; 544/166; 544/393; 549/452; 560/43; 560/125; 564/157

(57) ABSTRACT

A method of conferring a cooling effect on the skin or mucous membranes by applying thereto at least one compound of the Formula I:

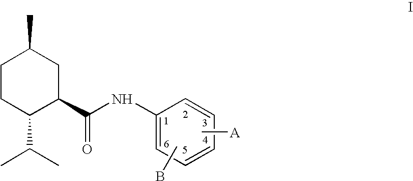

(a) wherein B is selected from H, CH₃, C₂H₅, OCH₃, OC₂H₅; and OH; and
(b) wherein A is a moiety of the formula —CO-D, wherein D is selected from the following moieties:
(i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring;
(ii) —NHCH₂COOCH₂CH₃, —NHCH₂CONH₂, —NHCH₂CH₂OCH₃, —NHCH₂CH₂OH, —NHCH₂CH(OH)CH₂OH and
(iii) a moiety selected from the group consisting of:

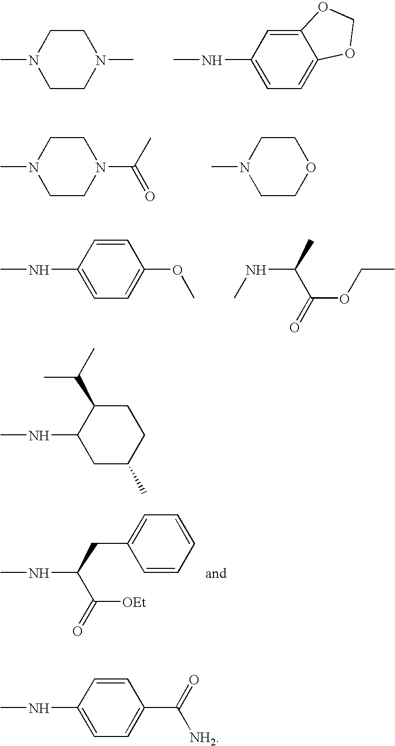

The compounds are useful for providing cooling effects in a variety of products, such as foodstuffs, confectionery, tobacco products, beverages, cosmetics, dentifrices, medicinal preparations, mouthwashes and toiletries.

This invention relates to cooling compounds.

Cooling compounds, that is, chemical compounds that impart a cooling sensation to the skin or the mucous membranes of the body, are well known to the art and are widely used in a variety of products such as foodstuffs, confectionery, tobacco products, beverages, cosmetics, dentifrices, medicinal preparations, mouthwashes and toiletries.

One class of cooling compounds that has enjoyed substantial success consists of N-substituted p-menthane carboxamides.

It has now been found that certain compounds exhibit a cooling effect that is both surprisingly strong and long-lasting. Therefore, a method is provided, which provides a cooling effect to the skin or mucous membranes and which comprises the application thereto of at least one compound of formula I:

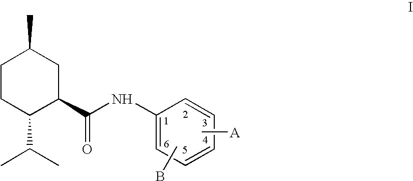

(a) wherein B is selected from H, CH₃, C₂H₅, OCH₃, OC₂H₅; and OH; and
(b) wherein A is a moiety of the formula —CO-D, wherein D is selected from the following moieties:
   (i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring;
   (ii) —NHCH₂COOCH₂CH₃, —NHCH₂CONH₂, —NHCH₂CH₂OCH₃, —NHCH₂CH₂OH, —NHCH₂CH(OH)CH₂OH and
   (iii) a moiety selected from the group consisting of:

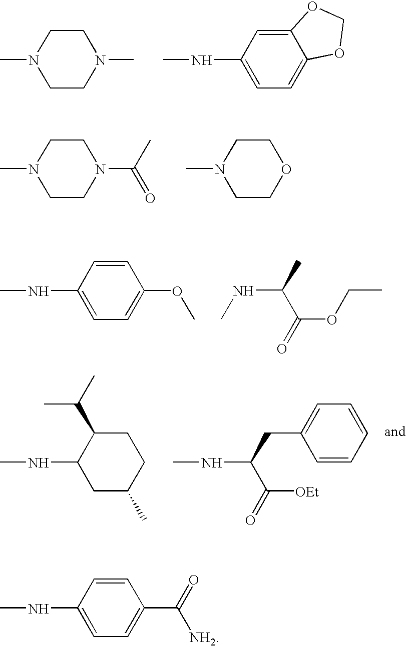

A product is provided that provides a cooling effect to the skin or mucous membranes, comprising at least one compound of Formula I:

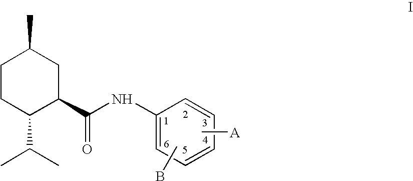

(a) wherein B is selected from H, CH₃, C₂H₅, OCH₃, OC₂H₅; and OH; and
(b) wherein A is a moiety of the formula —CO-D, wherein D is selected from the following moieties:
   (i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring;
   (ii) —NHCH₂COOCH₂CH₃, —NHCH₂CONH₂, —NHCH₂CH₂OCH₃, —NHCH₂CH₂OH, —NHCH₂CH(OH)CH₂OH and
   (iii) a moiety selected from the group consisting of:

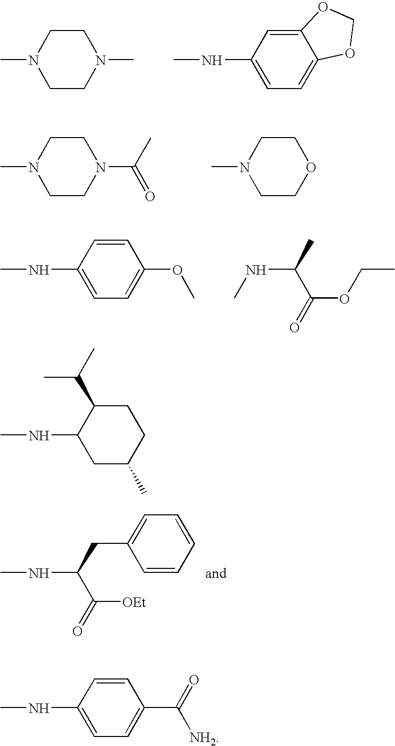

A compound which comprises formula I is provided:

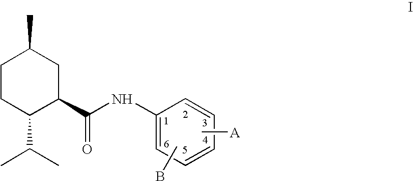

(a) wherein B is selected from H, CH₃, C₂H₅, OCH₃, OC₂H₅; and OH; and
(b) wherein A is a moiety of the formula —CO-D, wherein D is selected from the following moieties:
   (i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring, with the proviso that, when B is H and A is —CONH₂, A is attached either to the 2- or the 6-position of the phenyl ring;
   (ii) —NHCH₂COOCH₂CH₃, —NHCH₂CONH₂, —NHCH₂CH₂OCH₃, —NHCH₂CH₂OH, —NHCH₂CH(OH)CH₂OH and
   (iii) a moiety selected from the group consisting of:

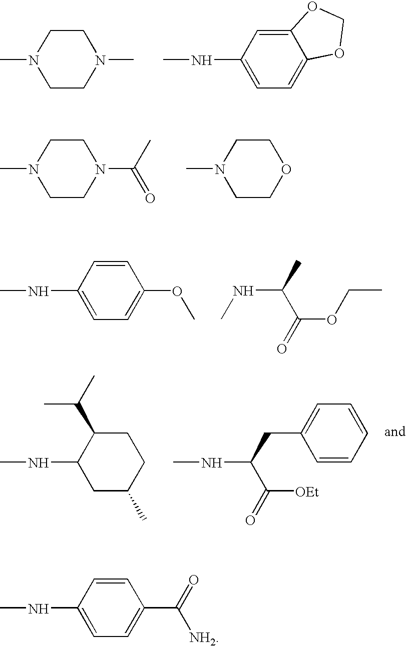

According to certain embodiments:
A is —CONH₂; and
A is in the 4-position and A is —CONHR³, R³ being selected from a C₁-C₄ straight or branched-chain aliphatic, alkoxyalkyl or hydroxyalkyl.
   According to other embodiments:
A is —CONH₂;
A is in the 4-position and A is —CONHR³, R³ being selected from a C₁-C₄ straight or branched-chain aliphatic, alkoxyalkyl or hydroxyalkyl; and
Bis H.

The compounds may be easily prepared and isolated by art-recognized methods.

Some of the compounds hereinabove described are available in a number of stereochemical forms. All possible stereochemical forms of the compounds hereinabove mentioned are encompassed by the scope of this invention.

They are distinguished from other cooling compounds of the prior art by their surprisingly high cooling effect (up to 10 times higher than that of known compounds) and by the longevity of the cooling effect, which adds to their attractiveness in a large variety of products.

For example, a small group of panelists was asked to taste various solutions of cooling compounds and indicate which solutions had a cooling intensity similar or slightly higher than that of a solution of menthol at 2 ppm. Results are shown in Table 1.

TABLE 1
experiment on cooling intensity and longevity

| Chemical | Concentration | |
|---|---|---|
| 1-Menthol | 2.0 | ppm |
| N-ethyl p-menthanecarboxamide (WS-3) | 1.5 | ppm |
| Formula I, B = H, A = 4-CONH₂ | 0.15 | ppm |
| Formula I, B = H, A = 3-CONH₂ | 0.2 | ppm |
| Formula I, B = H, A = 2-CONH₂ | 0.5 | ppm |
| Formula I, B = H, A = 4-CO-D, D = NHCH₂CH₂OCH₃ | 0.25 | ppm |
| Formula I, B = H, A = 4-CO-D, D = 4-methylpiperazine | 0.4 | ppm |

From Table 1, it can be seen that the compounds of Formula I are up to 10 times stronger than menthol, the comparative cooling compound. Compounds of Formula I are also much stronger than WS-3, the best cooling compound of the prior art.

The compounds provided may be used in a wide variety of products that are applied to the mouth or the skin to give a cooling sensation. Such products include, but are not limited to, foodstuffs, confectionery, tobacco products, beverages, cosmetics, dentifrices, medicinal preparations, mouthwashes, toiletries, and the like. By “applying” it is meant any form of bringing into contact, for example, oral ingestion or, in the case of tobacco products, inhalation. In the case of application to the skin, it may be, for example, by including the compound in a cream or salve, sprayable composition, or like composition. Therefore, also provided is a product that provides a cooling effect to the skin or mucous membranes, which product comprises at least one compound as hereinabove described.

In further embodiments, novel compounds are provided. Therefore, provided is a compound of the formula I

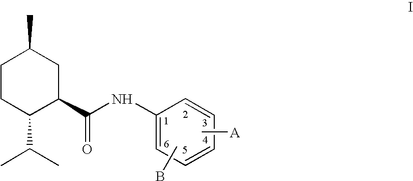

(a) wherein B is selected from H, CH₃, C₂H₅, OCH₃, OC₂H₅; and OH; and
(b) wherein A is a moiety of the formula —CO-D, wherein D is selected from the following moieties:
   (i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring, with the proviso that, when B is H and A is —CONH₂, A is attached either to the 2- or the 6-position of the phenyl ring;
   (ii) —NHCH₂COOCH₂CH₃, —NHCH₂CONH₂, —NHCH₂CH₂OCH₃, —NHCH₂CH₂OH, —NHCH₂CH(OH)CH₂OH, and
   (iii) a moiety selected from the group consisting of:

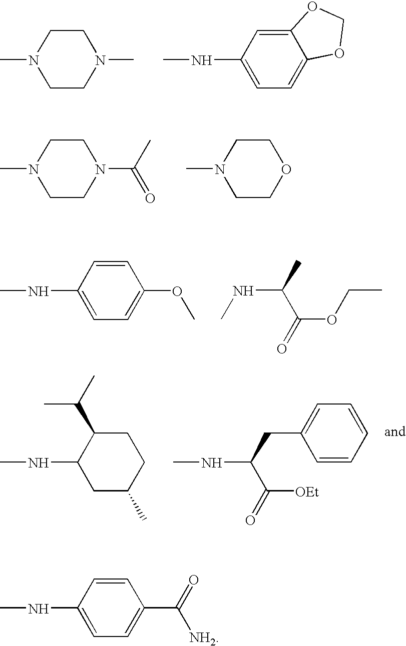

### EXPERIMENTAL

Listed below are non-limiting examples, which describe various embodiments.

### Example 1

### Preparation of N-(4-carboxamidophenyl) p-menthanecarboxamide

In a 4-L flask, 256 g of p-aminobenzamide and 156 g of pyridine were dissolved in 2.5 L of toluene. Under vigorous stirring, 586 g of solution of p-menthanecarboxyl chloride at ˜65% in toluene were added. The beige suspension was stirred vigorously overnight at room temperature. The suspension was filtered and the cake was washed with MTBE (tert-butyl methyl ether) and hot water. The product was recrystallized in ethanol to give 290 g of white crystals of the desired product with the following spectroscopic properties:

m.p.: 265-266° C.

¹H NMR (300 MHz; d₆-DMSO) δ: 9.86 (d, 1H), 7.6 (m, 3H), 7.5 (m, 2H), 7.01 (br. d, 1H), 2.87 (d, 1H), 2.3 (d, 1H), 2.14 (br. q, 2H) 1.81-1.32 (m, 5H), 1.16 (br. s, 1H), 0.89 (d, 3H), 0.8-0.1 (m, 6H)

¹³C NMR (75 MHz; d₆-DMSO) δ: 174.1, 167.2, 141.7, 128.3, 128.0, 117.9, 48.5, 43.4, 34.0, 31.6, 28.2, 26.6, 23.3, 22.0, 21, 15.9. One peak around 39 ppm is buried in the signal of DMSO.

### Example 2

### Preparation of N-(3-carboxamidophenyl) p-menthanecarboxamide

A preparation similar to that described in example 1 gives the desired product with the following spectroscopic properties:

¹H NMR (300 MHz; CDCl₃) δ: 9.00 (br. s, 1H), 7.92 (s, 1H), 7.78 (d, 1H), 7.55 (d, 1H), 7.36 (t, 1H), 6.22 (br. s, 1H), 3.39-3.11 (m, 2H), 2.29 (m, 1H), 1.89-1.61 (m, 4H), 1.4-1.2 (m, 2H), 1.1-0.95 (m, 2H), 0.91 (d, 3H), 0.8 (d, 3H), 0.78 (d, 3H)

¹³C NMR (75 MHz; CDCl₃) δ: 174.6, 168, 138.3, 133.6, 128.9, 123.3, 122.7, 118.6, 49.9, 44.0, 39.2, 34.3, 32.1, 28.6, 23.7, 22, 21.0, 15.8

### Example 3

### Preparation of N-(2-carboxamidophenyl) p-menthanecarboxamide

A preparation similar to that described in example 1 gives the desired product with the following spectroscopic properties:

MS: 302([M^{+•}]), 163, 136, 119, 83

¹H NMR (300 MHz; CDCl₃) δ: 11.27 (s, 1H), 8.68 (d, 1H), 7.56 (d, 1H), 7.48 (t, 1H), 7.05 (t, 1H), 6.44 (br. s, 1H), 5.89 (br. s, 1H), 2.24 (td, 1H), 1.92 (d, 1H), 1.82-1.52 (m, 4H), 1.41 (br. s, 1H), 1.35 (quintuplet, 1H), 1.14-0.95 (m, 2H), 0.91 (d, 3H), 0.84 (d, 3H), 0.83 (d, 3H)

¹³C NMR (300 MHz; CDCl₃) δ: 175.0, 171.1, 140.2, 133.1, 127.1, 122.2, 121.3, 118.2, 51.4, 44.5, 39.3, 34.4, 32.0, 28.7, 23.8, 22.1, 21.1, 15.9.

### Example 4

### Preparation of 4-[p-menthanecarbonyl-amino]-N-(2-methoxy-ethyl)-benzamide

A preparation similar to that described in example 1 gives the desired product with the following spectroscopic properties:

MS: 360([M^{+•}]), 345, 328, 302, 286, 194, 162, 136, 120, 83

### Example 5

### Preparation of N-(4-(4-methylpiperazine-1-carbonyl)phenyl) p-menthanecarboxamide

A preparation similar to that described in example 1 gives the desired product with the following spectroscopic properties:

MS: 385 ([M^{+•}]), 370, 286, 120, 99, 83

### Example 6

Application in Chewing gum

| | | |
|---|---|---|
| Gum Base Flama-T* | 25.180 | g |
| Compound of example 1 | 0.100 | g |
| Peppermint oil | 1.000 | g |
| Corn Syrup | 17.220 | g |
| Sugar | 55.170 | g |
| Glycerine | 1.330 | g |

*Flama-T is a trademark of Cafosa gum, Barcelona (Spain)

All the ingredients are mixed in the prewarmed gum base. The mixture is spread in thick films, cooled down and cut in sticks. A gum stick is chewed by a panelist for 15 minutes and spit out. When chewed, an agreeable cooling sensation was felt in all areas of the mouth. When spit out, the cooling sensation becomes intense and lasts for several hours

### Example 7

Application in toothpaste

| | |
|---|---|
| Opaque toothgel | 99.500 g |
| Compound of example 3 | 0.500 g |
| as a 5% gel in Peppermint oil | |

The chemicals are mixed in the toothgel, a piece of toothgel was put on a toothbrush and a panelist's teeth were brushed. The mouth was rinsed with water and the water was spit out. An intense cooling sensation was felt by the panelist in all areas of the mouth. The cooling perception lasted for several hours.

It will be understood that the embodiment(s) described herein is/are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

1. A method of providing a cooling effect to the skin or mucous membranes, comprising the application thereto of at least one compound of formula I: 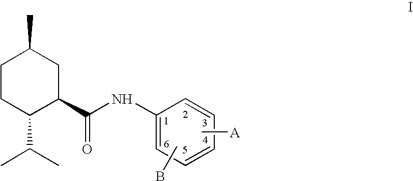
(a) wherein B is selected from H, CH₃, C₂Hs, OCH₃, OC₂H₅; and OH; and
(b) wherein A is a moiety of the formula —CO-D, wherein D is selected from the following moieties:
(i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring;
(ii) —NHCH₂COOCH₂CH₃, —NHCH₂CONH₂, —NHCH₂CH₂OCH₃, —NHCH₂CH₂OH, —NHCH₂CH(OH)CH₂OH and
(iii) a moiety selected from the group consisting of:

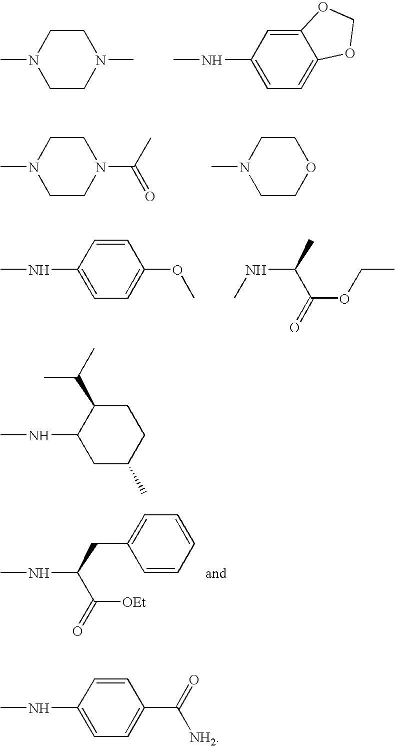

2. The method according to claim 1, wherein A is selected from the following:
A is CONH₂; and
A is in the 4-position and is —CONHR³, wherein R³ is selected from a C₁-C₄ straight or branched-chain aliphatic, alkoxyalkyl or hydroxyalkyl moiety.

3. The method according to claim 2, wherein B is H.

4. A product that provides a cooling effect to the skin or mucous membranes, comprising at least one compound of Formula I: 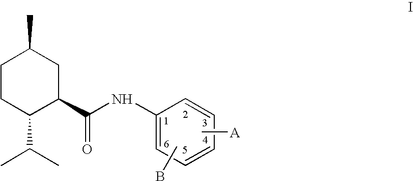
(a) wherein B is selected from H, CH₃, C₂Hs, OCH₃, OC₂H₅; and OH; and
(b) wherein A is a moiety of the formula —CO-D, wherein D is selected from the following moieties:
(i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring;
(ii) —NHCH₂COOCH₂CH₃, —NHCH₂CONH₂, —NHCH₂CH₂OCH₃, —NHCH₂CH₂OH, —NHCH₂CH(OH)CH₂OH and
(iii) a moiety selected from the group consisting of:

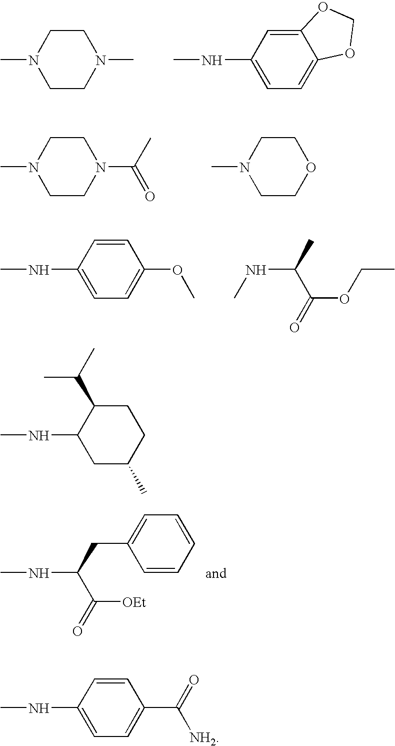

5. The product of claim 4, wherein A is selected from the following:
A is CONH₂; and
A is in the 4-position and is —CONHR³, in which R³ is selected from a C₁-C₄ straight or branched-chain aliphatic, alkoxyalkyl or hydroxyalkyl moiety.

6. The product of claim 5, wherein B is H.

7. The product of claim 4, wherein the product is at least one of foodstuffs, confectionery, tobacco products, beverages, cosmetics, dentifrices, medicinal preparations, mouthwashes or toiletries.

8. A compound comprising formula I: 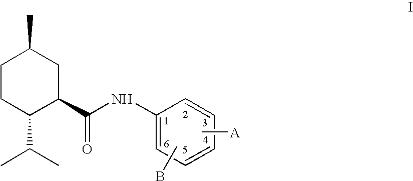
(a) wherein B is selected from H, CH₃, C₂H₅, OCH₃, OC₂H₅; and OH; and
(b) wherein A is a moiety of the formula —CO-D, wherein D is selected from the following moieties:
(i) —NR¹R², wherein R¹ and R² are independently selected from H and C₁-C₈ straight or branched-chain aliphatic, alkoxyalkyl, hydroxyalkyl, araliphatic and cycloalkyl groups, or R¹ and R² together with the nitrogen atom to which they are attached form part of an optionally-substituted, five- or six-membered heterocyclic ring, with the proviso that, when B is H and A is —CONH₂, A is attached either to the 2- or the 6-position of the phenyl ring;
(ii) —NHCH₂COOCH₂CH₃, —NHCH₂CONH₂, —NHCH₂CH₂OCH₃, —NHCH₂CH₂OH, —NHCH₂CH(OH)CH₂OH and
(iii) a moiety selected from the group consisting of:

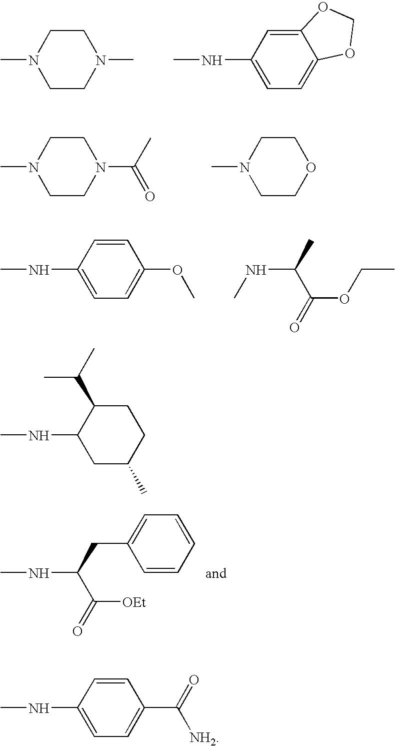

9. The compound of claim 8, wherein A is selected from the following:
A is CONH₂; and
A is in the 4-position and is —CONHR³, in which R³ is selected from a C₁-C₄ straight or branched-chain aliphatic, alkoxyalkyl or hydroxyalkyl moiety.

10. The compound of claim 9, wherein B is H.
